(19) United States
(12) Patent Application Publication
(10) Pub. No.: US 2023/0339747 A1
(43) Pub. Date: Oct. 26, 2023

(54) SYNGAS STAGE FOR CHEMICAL SYNTHESIS PLANT

(71) Applicant: TOPSOE A/S, Kgs. Lyngby (DK)

(72) Inventors: Kim AASBERG-PETERSEN, Allerød (DK); Thomas Sandahl CHRISTENSEN, Kgs. Lyngby (DK); Sudip DE SARKAR, Kokkedal (DK)

(73) Assignee: TOPSOE A/S, Kgs. Lyngby (DK)

(21) Appl. No.: 18/026,977

(22) PCT Filed: Oct. 12, 2021
(86) PCT No.: PCT/EP2021/078142

§ 371 Date: Mar. 17, 2023

(30) Foreign Application Priority Data
Oct. 14, 2020 (EP) 20201816.4

(51) Int. Cl. C01B 3/16; C01B 3/38; C10K 1/00; C25B 1/04; C07C 1/12; C07C 9/04; C25B 15/08

(52) U.S. Cl. CPC C01B 3/16; C01B 3/382; C10K 1/005; C25B 1/04; C07C 1/12; C07C 9/04; C25B 15/081; C01B 2203/0233; C01B 2203/0244; C01B 2203/0283; C01B 2203/0475; C01B 2203/062; C01B 2203/142; C01B 2203/148; C01B 2203/0866; C01B 2203/1235; C01B 2203/1614; C01B 2203/169; C01B 2203/1671

(57) ABSTRACT

A syngas stage, for use in a chemical plant, is provided, which includes a methanation section and an autothermal reforming section. The syngas stage makes effective utilization of CO2 rich stream and H2 rich stream. The syngas stage may include an external feed of hydrocarbons. A method for producing a syngas stream is also provided.

### TECHNICAL FIELD

The present invention relates to a syngas stage for use in a chemical synthesis plant, with effective use of various streams, in particular carbon dioxide. A method for producing a syngas stream is also provided. The syngas stage may or may not comprise an external feed of hydrocarbons. The syngas stage and method of the present invention provide overall better utilization of carbon dioxide.

### BACKGROUND

Carbon capture and utilization (CCU) has gained more relevance in the light of the rise of atmospheric CO₂ since the Industrial Revolution. In one way of utilizing CO₂, CO₂ and H₂ can be converted to synthesis gas (a gas rich in CO and H₂) which can be converted further to valuable products like alcohols (including methanol), fuels (such as gasoline, jet fuel, kerosene and/or diesel produced for example by the Fischer-Tropsch (F-T) process), and/or olefins etc.

Existing technologies focus primarily on stand-alone reverse Water Gas Shift (RWGS) processes to convert CO₂ and H₂ to synthesis gas. The synthesis gas can subsequently be converted to valuable products in the downstream processes as outlined above. The reverse water gas shift reaction proceeds according to the following reaction:

CO₂+H₂↔CO+H₂O (1)

The RWGS reaction (1) is an endothermic process which requires significant energy input for the desired conversion. Very high temperatures are needed to obtain sufficient conversion of carbon dioxide into carbon monoxide to make the process economically feasible. Undesired by-product formation of for example methane may also take place. High conversions of carbon dioxide can evidently also be obtained by high H₂/CO₂-ratio. However, this will often result in a synthesis gas with a (much) too high H₂/CO-ratio for the downstream synthesis. Furthermore, the hydrogen production costs will also increase considerably with a higher ratio.

Technologies relying on the RWGS reaction have other challenges. In some cases, hydrocarbon streams may be available as co-feed and/or the CO₂ or H₂ may comprise smaller amounts of hydrocarbons. An example is the availability of hydrocarbons from a downstream synthesis stage, where syngas gas from said syngas stage is converted to products (e.g. a propane and butane rich stream from an F-T stage; tail gas comprising different hydrocarbons from an F-T stage; naphtha stream from an F-T stage; propane and butane rich stream from a gasoline synthesis stage). Such hydrocarbons cannot be processed in an RWGS reactor. If the hydrocarbon streams from the downstream synthesis stage are not used at least in part for additional production of synthesis gas, the overall process may not be feasible from an economic point of view.

Another challenge exists with an RWGS reactor. CO₂ and H₂ are converted into a gas mixture comprising CO. CO may lead to carbon formation for example according to the following reaction:

CO+H₂→C+H₂O (2)

Carbon formation from carbon monoxide (2) may occur either on the catalyst or on the inner walls of the reactor. In the latter case the carbon formation may also be in the form of a corrosion type known as metal dusting. Carbon formation and metal dusting would typically take place at low to moderate temperatures up to 600-800° C. depending upon operating conditions, feed gas composition, feed temperature etc. The possibility of carbon formation or metal dusting is due to a relatively high concentration of carbon monoxide in a reactor with (only) reverse water gas shift taking place.

To address problems with existing technologies, a novel process of syngas preparation from primarily CO₂, H₂ and O₂ feed is presented in this document. The proposed layout has at least the following advantages:
1. CO₂, H₂, and O₂ can be converted to syngas with a desired H₂:CO ratio, even without using any external hydrocarbon feed to the syngas stage.
2. Utilization of any hydrocarbons either present in the feedstock or external to the syngas stage (e.g. recycled from a downstream synthesis stage)
3. A higher utilization of the carbon dioxide feed is possible compared to a stand-alone RWGS section. One particular aim is to utilize more CO₂ feed instead of external hydrocarbon feed as a source of carbon.
4. There is no risk of carbon formation or metal dusting from carbon monoxide
5. If an electrolyser is used as part of—or the entire source of—the hydrogen feed to the process, part or all of the oxygen, generated in the electrolyser along with H₂, can be used as an oxygen source that is required in the proposed process layout.

### SUMMARY

In a first aspect, a syngas stage (A) is provided, said syngas stage (A) comprising a methanation section (I) and an autothermal reforming (ATR) section (II).

The syngas stage further comprises:
a first feed comprising hydrogen to the syngas stage (A);
a second feed comprising carbon dioxide to the syngas stage (A);
a third feed comprising oxygen to the ATR section;
   wherein said syngas stage (A) is arranged to provide a syngas stream from said first, second and third feeds.

A method for producing a syngas stream, using the above-described syngas stage, is also provided.

Further details of the syngas stage and the method are specified in the following detailed descriptions, figures and claims.

### FIGURE LEGENDS

FIGS. 1-3 illustrate schematic layouts of various embodiments of the invention

FIG. 4 illustrates consumption of H₂ and O₂ feed vs. methanation section outlet CH₄ concentration.

### DETAILED DISCLOSURE

Unless otherwise specified, any given percentages for gas content are % by volume.

### Specific Embodiments

As set out above, a synthesis gas (syngas) stage is provided. The syngas stage comprises a methanation section and an autothermal reforming (ATR) section.

The syngas stage comprises various feeds.

The term “reactor(s)” is used interchangeably with the term “unit(s)”.

By the term “external” is meant “external to the syngas stage”.

A first feed comprising hydrogen is provided to the syngas stage. Suitably, the first feed consists essentially of hydrogen. The first feed of hydrogen is suitably “hydrogen rich” meaning that the major portion of this feed is hydrogen; i.e. over 75%, such as over 85%, preferably over 90%, more preferably over 95%, even more preferably over 99% of this feed is hydrogen. One source of the first feed of hydrogen can be an electrolyser stage. In addition to hydrogen the first feed may for example comprise steam, nitrogen, argon, carbon monoxide, carbon dioxide, and/or hydrocarbons. The first feed suitably comprises only low amounts of hydrocarbon, such as for example less than 5% hydrocarbons or less than 3% hydrocarbons or less than 1% hydrocarbons.

A second feed comprising carbon dioxide is provided to the syngas stage. Suitably, the second feed consists essentially of CO₂. The second feed of CO₂ is suitably “CO₂ rich” meaning that the major portion of this feed is CO₂; i.e. over 75%, such as over 85%, preferably over 90%, more preferably over 95%, even more preferably over 99% of this feed is CO₂. One source of the second feed of carbon dioxide can be one or more exhaust stream(s) from one or more chemical plant(s) or other plants such as cement plants or steel plants. One source of the second feed of carbon dioxide can also be carbon dioxide captured from one or more process stream(s) or atmospheric air. Another source of the second feed could be CO₂ captured or recovered from the flue gas for example from fired heaters, steam reformers, and/or power plants. The first and second feeds could be mixed before being added to the syngas stage. The second feed may in addition to CO₂ comprise for example steam, oxygen, nitrogen, oxygenates, amines, ammonia, carbon monoxide, and/or hydrocarbons. The second feed suitably comprises only low amounts of hydrocarbon, such as for example less than 5% hydrocarbons or less than 3% hydrocarbons or less than 1% hydrocarbons.

The ratio of H₂:CO₂ provided at the syngas stage inlet varies from 1.0-9.0, preferably 2.5-8.0, more preferably 2.5-4.5. The actual ratio will depend upon the desired end product downstream the synthesis stage.

In one aspect, when the synthesis gas is to be used for producing fuels in a downstream FT synthesis stage, the desired H₂/CO-ratio of the synthesis gas will typically be around 2.0. Using a simplistic view, one unit of hydrogen is needed to convert each unit of CO₂ into CO. The addition of O₂ will also require some hydrogen and furthermore hydrogen will be needed as source of energy for auxiliary purposes such as for example generation of power. All in all, this means that for an FT synthesis stage the H₂:CO₂-ratio at the syngas stage inlet should be in the range of 3.0-7.0 or more preferably from 3.0-6.0 and most preferably 3.0-5.0. If the desired end product is methanol or gasoline (via synthesis of methanol and the methanol-to-gasoline route) a similar consideration can be made and also in these cases the H₂:CO₂-ratio at the syngas stage inlet should be in the range of 3.0-7.0 or more preferably from 3.0-6.0 and most preferably 3.0-5.0.

It should be noted that in some cases H₂:CO₂ ratios less than 3.0 such as between 2.0-3.0 can be utilized.

A third feed comprising oxygen is provided to the ATR section. Suitably, the third feed consists essentially of oxygen. The third feed of O₂ is suitably “O₂ rich” meaning that the major portion of this feed is O₂; i.e. over 75% (dry) such as over 90% (dry) or over 95%, such as over 99% (dry) of this feed is O₂. This third feed may also comprise other components such as nitrogen, argon, and/or CO₂. This third feed will typically include a minor amount of steam (e.g. 5-10%. The source of third feed, oxygen, can be at least one air separation unit (ASU) and/or at least one membrane unit. The source of oxygen can also be an electrolyser stage. A part or all of the first feed, and a part or all of the third feed may come from at least one electrolyser stage. An electrolyser stage comprises a unit for converting steam or water into hydrogen and oxygen by use of electrical energy. Steam may be added to the third feed comprising oxygen, upstream the ATR section.

Optionally, the syngas stage comprises a fourth feed comprising hydrocarbons to said ATR section (II) and/or to said methanation section (I). The fourth feed may additionally comprise other components such as CO₂ and/or CO and/or H₂ and/or steam and/or other components such as nitrogen and/or argon. Suitably, the fourth feed consists essentially of hydrocarbons. The fourth feed of hydrocarbons is suitably “hydrocarbon rich” meaning that the major portion of this feed is hydrocarbons; i.e. over 50%, e.g. over 75%, such as over 85%, preferably over 90%, more preferably over 95%, even more preferably over 99% of this feed is hydrocarbons. The concentration of hydrocarbons in this fourth feed is determined prior to steam addition (i.e. determined as “dry concentration”).

In one aspect, the fourth feed is fed to the syngas stage, directly upstream of said ATR section (i.e. without any intervening stage). A “stage” comprises one or more “sections” which perform a change in the chemical composition of a feed, and may additionally comprise elements such as e.g. heat exchanger, mixer or compressor, which do not change the chemical composition of a feed or stream.

An example of such fourth feed can also be a natural gas stream external to the syngas stage. In one aspect, said fourth feed comprises one or more hydrocarbons selected from methane, ethane, propane or butanes.

The source of the fourth feed comprising hydrocarbons is external to the syngas stage. Possible sources of a fourth feed comprising hydrocarbons external to the syngas stage include natural gas, LPG, refinery off-gas, naphtha, off-gas, tail gas, purge gas, and renewables, but other options are also conceivable. Some of the sources of a fourth feed comprising hydrocarbons, such as e.g. tail gas or purge gas, may comprise less than 50% in hydrocarbons, typically in the range from 10-60% such as between 15 and 40%. The tail gas could for example come from a downstream FT-synthesis stage as described below. Such tail gas from an FT unit typically comprises between 10 and 40% hydrocarbons where methane typically is the hydrocarbon with the highest concentration.

In some cases, a feed comprising hydrocarbons may be subjected to prereforming before being provided to the syngas stage as the fourth feed. For example, when the fourth feed is e.g. a LPG and/or a naphtha stream (for example recycled from a downstream synthesis stage) or a natural gas feed, the syngas stage may further comprise a pre-reforming section, arranged in the fourth feed, upstream the syngas stage.

In a prereforming step, the following (endothermic) steam reforming reaction (3) and methanation reaction (4) (exothermic) take place to convert higher hydrocarbons. Additional water gas shift takes place through reaction (1):

CₙHₘ+nH₂O↔nCO+(n+m/2)H₂ (where n≥2, m≥4). (3)

CO₂+4H₂↔CH₄+2H₂O (4)

The prereformer outlet stream will comprise CO₂, CH₄, H₂O, and H₂ along with typically lower quantities of CO and possible other components. The prereforming step typically takes place at 350-600° C. or more preferably between 400 and 550° C. Steam is added to the hydrocarbon feed stream upstream the prereforming step. The prereforming step may take place either adiabatically or in a heated reactor, filled with catalysts including but not limited to Ni-based catalysts. Heating of the prereformer can be achieved by means of hot gas (e.g. ATR effluent gas) or in a heating section for example using an electrical heater or a fired heater. Hydrogen or other combustible components may be used to obtain the necessary heat input.

A hydrocarbon stream may also contain olefins. In this case the olefins may be subjected to hydrogenation to the corresponding paraffins before addition to a prereformer or the syngas stage as the fourth feed.

In some cases, the hydrocarbons contain minor amount of poisons, such as sulfur. In this case, the hydrocarbons may be subjected to the step or steps of purification such as desulfurization.

The fourth feed may comprise one or more streams comprising hydrocarbons that are either mixed or added separately to the syngas stage.

Optionally, a hydrocarbon-containing off-gas stream (from the synthesis stage) may be fed to the syngas stage (e.g. to the ATR section or to the methanation section) as all or part of the fourth feed comprising hydrocarbons. The source of fourth feed can be part or all of a stream comprising hydrocarbons produced in a synthesis stage. A number of recycle streams may be added to various points of the synthesis gas stage—there may either be mixed or added separately—in other words this fourth feed may be several separate or mixed streams.

In yet another possibility, fourth feed can be a so-called tail gas from a Fisher-Tropsch unit. This tail gas typically comprises CO₂, CO, H₂, methane and olefins. The tail gas may comprise hydrocarbons typically in the range from 10-60% such as between 15 and 40% as described above.

### Syngas Stage

The syngas stage is arranged to provide a syngas stream (from at least said first, second third feeds). For the avoidance of doubt, the terms “syngas” and “synthesis gas” are synonymous. Furthermore, the term “provide a syngas stream” in this context must be understood as to “produce a syngas stream”.

The syngas stage comprises a methanation section and an autothermal reforming (ATR) section. The syngas stage may comprise additional sections as required. Various sections will be described in the following.

### ATR Section

The syngas stage comprises an autothermal reforming (ATR) section. The ATR section may comprise one or more autothermal reactors (ATR). The key part of the ATR section is the ATR reactor. All feeds to the ATR section are preheated as required and/or received from the methanation section. The ATR reactor typically comprises a burner, a combustion chamber, and a catalyst bed contained within a refractory lined pressure shell. In an ATR reactor, partial combustion of the hydrocarbon containing feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions. More details of ATR and a full description can be found in the art such as “Studies in Surface Science and Catalysis, Vol. 152,” Synthesis gas production for FT synthesis”; Chapter 4, p.258-352, 2004”.

Typically, the effluent gas from the ATR reactor has a temperature of 900-1100° C. The effluent gas normally comprises H₂, CO, CO₂, and steam. Other components such as methane, nitrogen, and argon may also be present often in minor amounts. The operating pressure of the ATR reactor will be between 5 and 100 bars or more preferably between 15 and 60 bars.

The syngas stream from the ATR reactor is cooled in a cooling train normally comprising a waste heat boiler(s) (WHB) and one or more additional heat exchangers. The cooling medium in the WHB is (boiler feed) water which is evaporated to steam. The syngas stream is further cooled to below the dew point for example by preheating the utilities and/or partial preheating of one or more feed streams and cooling in air cooler and/or water cooler. Condensed H₂O is taken out as process condensate in a separator to provide a syngas stream with low H₂O content, which is sent to the synthesis stage.

### Methanation Section

In one aspect, the syngas stage comprises or consists of a methanation section, which is preferably arranged upstream the ATR section. The methanation section is in fluid connection with said ATR section. A part or all of the first feed may be fed to the methanation section; and a part or all of the second feed may be fed to the methanation section.

The heat generated in the methanation process obviates completely or reduces significantly the need for external preheating of the feed to the autothermal reforming section as is the case for traditional natural gas-based plants with autothermal reforming. Such external preheating may for example take place in a fired heater. The required heat in such a fired heater is generated by combustion of for example hydrogen and/or a hydrocarbon. In the former case this consumes part of the feed and in the second case this leads to CO₂ emissions. Furthermore, a fired heater is an expensive piece of equipment which may also take up a considerable plot area.

Preheating of part or all of the first and or second feed in the methanation section may as described above be done by a fired heater. The preheating may also take place by other means such as an electrical heater or by using steam. The steam may for example be generated externally to the syngas stage or for example in the waste heat boiler in the ATR section as described above.

Another possibility of preheating the first and/or second feeds is by utilizing part or all of the syngas stream from the ATR reactor. In this embodiment part or all of the syngas stream from the ATR reactor is cooled by indirect heat exchange with the first and/or second feeds. This embodiment has the advantage that it avoids or reduces the use of fuel for a fired heater and/or the power for an electrical heater. In a similar fashion part or all of the preheating of the first and/or second feeds may be done by indirect heat exchange with the cooled syngas stream leaving the waste heat boiler downstream the ATR reactor.

In another possibility, a part of all of the preheating of first and/or second feeds may be done in indirect heat exchange with the effluent leaving one of the units in methanation section. In this case, methanation unit may comprise more than one methanation units or reactors. Each methanation unit can be either an adiabatic or heated reactor.

The term “preheat” means the heating of the first and/or second feed streams before these feed streams are directed to a methanation reactor in the methanation section.

Heating of any feed stream to any of the methanation reactor(s) in the methanation section may also be done by one or more of the methods just described.

The stream(s) leaving the methanation section and used as feed for the ATR section may also be heated by indirect heat exchange with part or all of the syngas stream leaving the ATR reactor. This also saves oxygen and/or reduces or eliminates the need for heating by a fired heater or an electrical heater.

As indicated earlier, state of art for producing a synthesis gas from CO₂ and hydrogen is based on selective RWGS with no methanation. Compared to this scheme, the combination of methanation and ATR has several advantages. This includes the possibility of utilizing external hydrocarbon feeds, such as recycle streams from a synthesis stage potentially arranged downstream the syngas stage. Furthermore, the outlet temperature from the ATR reactor in the ATR section will typically be in the range of 900-1100° C. This is in most cases higher than is possible with a stand-alone RWGS unit. This increases the production of carbon monoxide as this is thermodynamically favoured by higher temperatures. It should also be noted that even if methane is formed in the methanation section, the content of methane in the final synthesis gas sent to the synthesis stage is low due to the high exit temperature from the ATR reactor in the ATR section. Advantageously, the exit temperature from the ATR is between 1000-1100° C. This temperature range results in low (<20 vol %) levels of methane in the synthesis gas. Even higher temperatures will increase the oxygen consumption without significant other benefits.

It is an advantage for most applications that the content of methane in the synthesis gas sent to the synthesis stage is low. For most types of synthesis stages, methane is an inert or even a synthesis stage by-product. Hence, in one preferred embodiment, the content of methane in the synthesis gas sent to the synthesis stage is less than 5%, such as less than 3% or even less than 2%. In one preferred embodiment, the methane content in the gas leaving the methanation section (I) is arranged to be less than 20%, preferably less than 15% by volume (of the entire gas leaving the methanation section). This stream therefore comprises methane but is lean in methane as opposed to a methane rich stream. A low content of methane is advantageous as this reduces the amount of oxygen needed in the ATR section. In some cases the lower methane concentration may also reduce the overall amount of the first feed comprising hydrogen required.

In the methanation section both the reverse water gas shift (1) and the methanation reaction(s) takes place. The methanation reaction can be illustrated by the following reactions:

CO₂+4H₂↔CH₄+2H₂O (as per equation 4, above)

CO+3H₂↔CH₄+H₂O (5)

The reverse water gas shift reaction can be illustrated by the following:

CO₂+H₂↔CO+H₂O (as per equation 1, above)

The methanation section comprises reactor(s) or unit(s) that contain a catalyst active both for reverse water gas shift and methanation. The fact that methanation also takes place means that the concentration of carbon monoxide in reactors or units in the methanation section is lower than if only the reverse water gas shift was taking place. This avoids the high concentration of carbon monoxide and avoids or reduces significantly the risk of carbon formation and metal dusting.

It seems counterintuitive to insert a methanation section upstream an ATR section. In the methanation section methane is formed and a large part of the formed methane is then converted either in a later unit in the methanation section and/or in the ATR section. However, the inventors have found that the heat of methanation can be utilized for preheating the feed to the ATR section. This avoids or reduces the need for a dedicated feed preheater. Reducing the preheat duty will also reduce the required combustion to provide the required energy and thereby the emissions of CO₂ in case the preheater is a fired heater with hydrocarbon fuel. The methanation section may comprise or consist of one or more methanation units, arranged in series, such as two or more methanation units, three or more methanation units or four or more methanation units. In such methanation units, at least part of the CO₂ and H₂ are converted to methane, steam, and carbon monoxide. In other words, the effluent from a methanation unit comprises CO₂, H₂, CO, CH₄, and steam. Typically, the effluent gas from a methanation unit is at or close to chemical equilibrium considering reactions (1) and (4). This is also the case if methane or other hydrocarbons are present in the feed to a methanation unit. The methanation units may be adiabatic or the methanation units may also be heated. The effluent temperature from each methanation unit can be 250-900° C., preferably 600-850° C., more preferably 650-840° C., depending on the extent of methanation and the feed gas composition, and operating conditions etc. Parallel methanation units are also conceivable.

As mentioned above, hydrocarbons may be present in the first and/or second feed to the methanation section and/or a separate fourth feed may be added to the methanation section. In this case the hydrocarbons are also present in the feed to one or more methanation reactors. Methane reacts as follows in a methanation reactor:

CH₄+H₂O↔CO+3H₂ (6)

In case higher hydrocarbons are present in the feed to a methanation reactor, these react according to reaction (3) above.

In some cases, it may be desirable to adjust the operating temperatures in the methanation unit for example to limit the extent of deactivation of the catalyst due to sintering. This is especially the case if the methanation unit or methanation reactor is adiabatic. The highest temperature in an adiabatic methanation unit will normally be at the outlet. Hence, it may be desirable to control the exit temperature from one or more methanation units to for example a temperature in the range 600-750° C., such as about 650° C., 675° C., 700° C., or 725° C. This may be accomplished by controlling the feed streams to the individual methanation units in the methanation section, if more than one methanation unit is present. By controlling the molar ratios between the part of the first feed and the part of the second feed as well as the molar ratio between the part of the first feed and the part of the fifth feed (if present) added to a methanation unit, it is possible to control the exit temperature of an adiabatic methanation unit. The inlet temperature(s) of the feed streams can also be used for this purpose.

It is desirable to reduce the oxygen consumption in the ATR section as much as possible. This can be accomplished by a high exit temperature and/or a low methane content in the gas leaving the methanation section. This is different compared to what is normally desired in plants for production of methane using methanation where a methane rich stream is desired. In one embodiment, therefore the exit gas from the methanation section is a methane lean stream. Examples of a methane lean stream are a stream containing less than 20% by volume of methane such as less than 15% or even less than 12% by volume of methane. The units and operating conditions in the methanation section can be arranged to provide such a methane lean stream.

In plants for the production of methane, it has been found that it is desirable to have a relatively low inlet temperature to the methanation reactors to optimize the economics of methane production. However, the situation is different in the production of synthesis gas from CO₂ and H₂. As described above, it may be desirable to have a methane lean stream at the outlet of the methanation section. Hence, in one embodiment, the feed temperature to one or more of the methanation reactors may be above 350° C., such as above 375° C., or even above 400° C. This provides a relatively high exit temperature from a methanation reactor and an exit temperature with a relatively lean concentration of methane as described above.

In one embodiment the methanation section comprises or consists of one methanation reactor. In a specific embodiment this methanation reactor is adiabatic (except for possible heat loss in certain circumstances). In this embodiment the feed temperature to the methanation reactor is adjusted such that the exit temperature thereof is above 750° C., such as above 775° C. or above 800° C. In a particular embodiment the exit gas from this reactor is not actively cooled (except for heat loss and possible mixing with other streams in certain circumstances) before being fed to the ATR section.

In one embodiment the means are provided to adjust the feed temperature to one or more of the methanation reactors to obtain the desired exit temperature. It is recognized that methanation catalysts deactivate with time. In some cases, it may therefore be desirable to be able to increase the feed temperature to one or more methanation reactors to ensure that sufficient conversion takes place in the one or more methanation reactors for the duration of the catalyst lifetime. In a particular embodiment such means are provided to adjust the inlet temperature to the first methanation reactor, where said first methanation reactor is adiabatic.

In another embodiment the methanation section comprises or consists of two methanation reactors. In this embodiment at least part or all of the first feed and part or all of the second feed are directed to the first methanation reactor, wherein said first methanation reactor is preferably adiabatic. The effluent from this first methanation reactor is cooled and part or all of the water is condensed and removed. The remaining part of the effluent from the first methanation reactor is mixed with at least the remaining part of the first and/or second feed and directed to the second methanation reactor. The feed temperature to this second methanation reactor may preferably be 300-500° C. The effluent from the second methanation reactor is directed to the ATR section without any further active cooling. This embodiment with condensation of water has the advantage that the CO₂ in the synthesis gas leaving the ATR section is lower than if no water was removed.

In one embodiment the methanation section comprises a heated methanation reactor. In this case, the exit temperature from the methanation reactor is higher than if the reactor were adiabatic. This has the advantage of further reducing the methane content in the feed gas to the ATR section and decreasing the oxygen consumption.

In another embodiment part or all of a (or more) methanation reactor is heated.

Heating of a methanation reactor seems counterintuitive as the methanation reaction is exothermic. However, the methanation reactor may also be considered as part of the process for converting CO₂ and H₂ into CO by the endothermic reverse water gas shift reaction.

The heat for the heated methanation reactor may be provided for example by a fired heater or an electrical heater. Alternatively, the heat may be provided by cooling of part or all of the syngas leaving the ATR reactor by indirect heat exchange. The advantages of this embodiment are the same as described above regarding preheating of the first and/or second feeds.

In one embodiment, the methanation section comprises one adiabatic methanation reactor. In a specific embodiment the first feed of hydrogen is added to this adiabatic reactor together with only part of the second feed comprising carbon dioxide. Part or all of the fourth feed may optionally also be added to the feed to the adiabatic reactor. This limits the extent of methanation reducing the methane content in the feed to the ATR section and also limits the exit temperature from the methanation reactor. Preferably, this exit temperature is below 700° C., such as 650-700° C. This limitation has the advantage that the rate of catalyst deactivation by sintering is lower than if all the second feed was added to the adiabatic reactor.

In another embodiment the methanation section comprises two adiabatic reactors in series. In a specific embodiment the first feed of hydrogen is added to the first adiabatic reactor together with only part of the second feed comprising carbon dioxide. Part or all of the remaining part of the second feed comprising carbon dioxide is added to the second adiabatic reactor.

In another embodiment, the methanation section comprises an adiabatic methanation reactor followed by a heated methanation reactor. Part of the second feed of carbon dioxide bypasses the first adiabatic reactor and is instead fed to the heated methanation reactor.

In a specific embodiment (used in the examples), methanation section comprises or consists of two methanation units or reactors, where at least a part or all of the first feed and a part or all of the second feed are preheated, mixed and directed to the first methanation reactor, wherein the said methanation reactor is of adiabatic type. Preheating of the first and second feeds can be done by using steam, for example generated in the waste heat boiler after ATR reactor. Further preheating of mixed feed to first methanation reactor can be done using indirect heat exchange by partially cooling of first methanation unit effluent. Inlet temperature to the first methanation unit may preferably be 300-400° C. while effluent temperature may preferably 650-700° C. Partially cooled effluent from first methanation reactor is then mixed with remaining part of the preheated first and/or second feed and directed to the second methanation reactor, wherein the said methanation reactor is a heated reactor. The feed temperature to the second methanation reactor may preferably be 400-600° C., while the process gas outlet temperature from methanation section may preferably be 750-850° C. The process gas from methanation section, comprising less than 20 vol % methane and preferably less than 15 vol % methane, is then fed to ATR reactor along with third feed and optionally available fourth feed to produce a final syngas product stream, after cooling and separation of condensed water.

The control of the ratios of the various feed streams to the methanation units and the ratios of the various feed streams fed to the methanation section and directly to the methanation section may also be used to impact the synthesis gas composition.

Parts of the first feed comprising hydrogen may be fed separately to different methanation units in the methanation section; or the entire first feed comprising hydrogen may be fed together to the methanation unit located furthest upstream in the methanation section. Similarly, parts of the second feed comprising carbon dioxide may be fed separately to different methanation units in the methanation section; or the entire second feed comprising carbon dioxide may be fed together to the methanation unit located furthest upstream in the methanation section.

In a specific embodiment, all of the first feed comprising hydrogen is fed to the first of the methanation units together with part of the second feed comprising carbon dioxide. The remaining part of the carbon dioxide is distributed between the remaining methanation units and the exit temperature of the final methanation unit is between 650-900° C. such as between 750-850° C.

Additional H₂ feed and/or CO₂ feed can be added to different parts of the methanation section. For instance, part of the hydrogen or CO₂ feed could be provided to a second (or even third . . . ) methanation unit. Additionally, part of the effluent from one methanation unit can be (optionally) cooled and recycled to the inlet of said methanation unit and/or to the inlet of any additional methanation unit(s) located upstream said one methanation unit. Optionally, effluent from methanation section can be cooled below its dew point and a part of the water may be removed from this effluent before it is recycled to the inlet of the methanation unit or any upstream methanation unit.

A stream comprising H₂ and/or CO₂ may also be recovered from downstream the ATR section and be recycled to the methanation section. Addition of steam to the methanation section and/or between the methanation section and the ATR section may also occur.

In this aspect, the exothermic nature of the methanation reaction may be utilized for preheating the ATR feed. Some heating of the ATR section by external means may be either needed or desirable, for example for control purposes. Therefore, the reaction heat of the methanation reaction may only cause part of the temperature increase upstream the ATR section.

Normally, the RWGS (reaction (1) and/or the water gas shift reaction (reverse of reaction (1)) will also take place in the methanation unit. In many cases, the gas composition at the exit of each methanation unit will be at or close to chemical equilibrium with respect to the water gas shift/reverse water gas shift and the methanation reactions at the exit temperature and pressure of said methanation unit.

The methanation reaction (4) is very exothermic. In some cases, it is desirable to adjust the temperature at the outlet of a methanation unit or from the methanation section to a given value which may be in the range of 550-800° C. such as between 600-700° C. If part or all of a fourth feed comprising hydrocarbons is added to a methanation unit, this may reduce the exit temperature due to the fact that steam reforming (reverse of reaction (4) and/or reaction (3)) will take place.

If the effluent from a prereforming step is added to a methanation unit, the exit temperature from such methanation unit will typically be lower than if no such stream is added. The methane in the prereforming step effluent will react according to the endothermic steam reforming reaction:

CH₄+H₂O↔CO+3H₂ (reaction 6, above)

The presence of methane in the feed will limit the extent of the methanation reaction due to the chemical equilibrium.

The output from the methanation section is a stream comprising CO₂, H₂, CO, H₂O and CH₄.

In a particular aspect where the syngas stage is followed by a F-T synthesis stage, the tail gas from an FT synthesis stage will normally not be added to a methanation unit but fed directly to the ATR section. If excess tail gas from the FT synthesis stage is available, this may be hydrogenated and fed to the methanation section.

In one embodiment, the inlet temperature of at least one of the methanation units will be between 300-500° C.

The control of the ratios of the various feed streams to the methanation units and the ratios of the various feed streams fed to the methanation section and directly to the methanation section may also be used to impact the synthesis gas composition.

The extent of methanation (and thereby the composition of the gas to the ATR section) depends on a number of factors including the ratio of the feed streams to the methanation section and the inlet and exit temperature to and from each methanation unit and the extent of water removal (if any) from the methanation section. For a given gas composition and temperature of the gas to the ATR section, the synthesis gas from the ATR depends upon the amount of oxygen added. Increasing the amount of oxygen increases the ATR reactor exit temperature and thereby reduces the H₂/CO-ratio.

In another embodiment, the syngas stage (A) comprises a methanation section (I) arranged in parallel to said ATR section (II). At least a portion of the first feed and at least a portion of the second feed are arranged to be fed to the methanation section (I) and said methanation section (I) is arranged to convert said at least a portion of the first feed and at least a portion of the second feed to a first syngas stream. A third feed of oxygen is arranged to be fed to the ATR section (II); and wherein said ATR section (II) is arranged to convert part or all of the hydrocarbon streams and said third feed comprising oxygen—along with the remaining portions of the first and second streams—to a second syngas stream. The first syngas stream from the methanation section (I) is arranged to be combined with the second syngas stream from the ATR section (II).

Compared to in series methanation and ATR section, this embodiment reduces the amount of oxygen needed.

In one embodiment, the syngas stream has a (H₂−CO₂)/(CO+CO₂) ratio in the range 1.50-2.50; preferably 1.80-2.30, more preferably 1.90-2.20. Such ratio is desirable for example if the syngas is to be used for methanol synthesis. In another embodiment, the (H₂/CO)-ratio is adjusted to 1.8-2.1. Such ratio is advantageous in case the syngas is to be used for a downstream Fischer-Tropsch synthesis.

### Post ATR CO₂-Conversion Unit

In another aspect, the unit comprises a post-conversion (post-ATR conversion, PAC) unit or reactor, located downstream the ATR section.

The PAC unit may be either adiabatic or a heated reactor using for example a Ni-based catalyst and/or a catalyst with noble metals such as Ru, Rh, Pd, and/or Ir as the active material. In such a PAC unit, a stream comprising carbon dioxide such as part of the second feed and part or all of the syngas from the ATR section is mixed and directed to the PAC unit. The mixed stream is converted to a syngas with higher carbon monoxide content via both reactions (4) and (1)—above—in the PAC unit. Reactions (4) and (1) will typically be at or close to chemical equilibrium at the outlet of the PAC unit. The effluent from the PAC section is a stream comprising CO₂, H₂, CO, H₂O and CH₄. The PAC effluent temperature from each PAC unit can be 700-1000° C., preferably 800-950° C., more preferably 850-920° C. The advantage of the PAC unit is the ability to produce a synthesis gas a lower H₂/CO-ratio compared to the effluent stream from the ATR section. Furthermore, the fact that a stream comprising carbon dioxide such as part of the second feed is directed to the PAC unit (such as an adiabatic PAC unit) instead of to the ATR section, reduces the size of the ATR section. This may in some cases reduce the overall cost.

The effluent stream from the PAC unit is cooled as described above to provide a syngas stream for the synthesis stage.

This CO₂-conversion (PAC) unit may be included in any of the aspects described above.

### Synthesis Stage

The syngas stage may provide a syngas stream to a synthesis stage. The synthesis stage is typically arranged to convert the syngas stream into at least a product stream. Often a hydrocarbon-containing off-gas stream is generated in the synthesis stage. Suitably, at least a portion of said hydrocarbon-containing off-gas stream is fed to the syngas stage as a fourth feed, upstream of said ATR section and preferably between said methanation section and said ATR section.

As noted, the syngas stage might comprise an external hydrocarbon feed such as, any recycle stream(s) from the synthesis stage.

Examples of the synthesis stage are a Fischer-Tropsch synthesis (F-T) stage or a methanol synthesis stage.

### Electrolyser Stage

The syngas unit may further comprise an electrolyser stage arranged to convert water or steam into at least a hydrogen-containing stream and an oxygen-containing stream, wherein at least a part of said hydrogen-containing stream from the electrolyser stage is fed to the syngas stage as said first feed and/or wherein at least a part of said oxygen-containing stream from the electrolyser stage is fed to the syngas stage as said third feed. An electrolyser stage may comprise one or more electrolysis units, for example based on solid oxide electrolysis.

At least a part of the hydrogen-containing stream from the electrolyser stage may be fed to the syngas stage as said first feed. Alternatively, or additionally, at least a part of the oxygen-containing stream from the electrolyser stage is fed to the syngas stage as said third feed. This provides an effective source of the first and third feeds.

In a preferred aspect, all of the hydrogen in the first feed and all of the oxygen in the third feed is produced by electrolysis. In this manner the hydrogen and the oxygen required by the syngas stage is produced by steam and electricity. Furthermore, if the electricity is produced only by renewable sources, the hydrogen and oxygen in the first and third feed, respectively, are produced without fossil feedstock or fuel.

Preferably, the water or steam fed to the electrolyser stage is obtained from one or more units or stages in said syngas stage. The use of an electrolyser stage may be combined with any of the described embodiments in this document.

### Additional Aspects

The composition of the syngas from the syngas stage can be adjusted in other ways. For instance, the plant may further comprise a carbon dioxide removal section, located downstream said syngas stage, and arranged to remove at least part of the carbon dioxide from the syngas stream. In this case, at least a portion of the carbon dioxide removed from the syngas stream in said carbon dioxide removal section, and may be compressed and fed as part of said second feed to the syngas stage. Carbon dioxide removal units can be, but not limited to, an amine-based unit or a membrane unit or a cryogenic unit or a pressure or temperature swing adsorption unit. If the synthesis stage is a Fischer-Tropsch stage, the removal of CO₂ has the advantage that this reduces the inert content of the feed gas to the FT-stage. Recycling the unconverted CO₂ to the syngas stage such as to the methanation section and/or the ATR section has the advantage of increasing the overall carbon efficiency of the plant.

Furthermore, the plant may further comprise a hydrogen removal section, located downstream said syngas stage, and arranged to remove at least part of the hydrogen from the syngas stream. In this case, at least a portion of the hydrogen removed from the syngas stream in said hydrogen removal section may be compressed and fed as said part of said first feed to the syngas stage. Hydrogen removal units can be, but not limited to, pressure swing adsorption (PSA) units or membrane units. If the synthesis stage is a FT stage, the removal of hydrogen can be used to adapt the H₂/CO ratio in the feed gas to the synthesis stage to the desired value of ca. 2. Recycling of the hydrogen to the methanation section or the ATR section may reduce the required amount of the first feed comprising hydrogen.

An off-gas stream external to the syngas stage, may be treated to remove one or more components, or to change the chemical nature of one or more components, prior to being fed to the syngas stage. The off-gas, for example when it is an F-T tail gas, may comprise olefins. Olefins increase the risk of carbon deposition and/or metal dusting at high temperatures. Therefore, the plant may further comprise a hydrogenator arranged in the F-T tail gas recycle stream. The hydrogenator arranged to hydrogenate the fourth feed, before said fourth feed enters the syngas stage. In this manner, olefins can effectively be converted to saturated hydrocarbons before entering the syngas stage.

An off-gas stream or the part of an off-gas stream not recycled to the synthesis gas stage or used for other purposes may be used to produce additional synthesis gas in a separate synthesis gas generator. Such a synthesis gas generator may comprise technologies known in the art such as ATR, steam reforming (SMR), and/or adiabatic prereforming, but also other technologies are known. Such additional synthesis gas may be fed to the synthesis stage. As an example, tail gas from a Fischer-Tropsch synthesis stage may be converted into additional synthesis gas by means known in the art such as comprising hydrogenation, followed by water gas shift, and autothermal reforming.

### Method

A method for producing a syngas stream is provided, said method comprising the steps of:
providing a syngas stage as defined herein;
supplying a first feed comprising hydrogen to the syngas stage;
supplying a second feed comprising carbon dioxide to the syngas stage;
supplying a third feed comprising oxygen to the ATR section;
optionally, supplying a fourth feed comprising hydrocarbons to said methanation section (I) and/or to said ATR section (II); and
converting said first, second, third and—optionally, fourth—feeds in said syngas stage to a syngas stream.

All aspects relating to the syngas stage set out above are equally applicable to the method using said syngas stage. In particular, the following aspects of particular importance are noted:
an electrolyser stage may be located upstream the syngas stage and the method may further comprise conversion of water or steam into at least a hydrogen-containing stream and an oxygen-containing stream. The method may further comprise the steps of; feeding at least a part of said hydrogen-containing stream from the electrolyser stage to the syngas stage as part or all of said first feed of hydrogen and/or feeding at least a part of said oxygen-containing stream from the electrolyser stage to the syngas stage as part or all of said third feed of oxygen. The method may further comprise obtaining the water or steam which is fed to the electrolyser stage is obtained as condensate or steam from one or more units in the syngas stage.
where the plant comprises a methanation section (I) and an ATR section (II) it is preferred that no water condensation takes place in the methanation section (I).
the methanation section (I) may comprise or consist of one or more adiabatic methanation units, wherein the temperature of the gas at the exit of the adiabatic methanation unit is greater than 700° C.
the methanation section (I) may comprise or consist of one or more adiabatic methanation units, and wherein no active cooling of the gas exiting the adiabatic methanation unit takes place before said gas is directed to the ATR section (II).
the methanation section (I) may comprise or consist of one or more methanation units, such as two or more methanation units and wherein the gas temperature at the inlet to the first methanation unit in the methanation section is >350° C.; such as >400° C.
if a CO₂ removal stage is arranged downstream the ATR section (II) CO₂ may be removed from the syngas stream by means of said CO₂ removal stage, and a part or all of the recovered CO₂ may be recycled to syngas stage as a part of second feed comprising CO₂
the methane content in the gas leaving the methanation section (I) is suitably less than 20%, preferably less than 15% by volume.

### DETAILED DESCRIPTION OF THE FIGURES

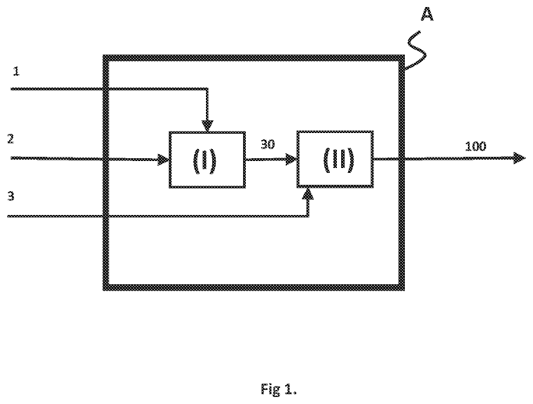

FIGS. 1-3 illustrate schematic layouts of embodiments of the invention.

In FIG. 1:
A syngas stage
I methanation section
II autothermal reforming section
1 first feed (comprising hydrogen) to syngas stage (A)
1′ a part of first feed (comprising hydrogen) from electrolysis stage
2 second feed (comprising carbon dioxide) to syngas stage (A)
3 third feed (comprising oxygen) to syngas stage (A)
4 fourth feed (comprising hydrocarbon) to syngas stage (A)
5 fifth feed (comprising steam) to syngas stage (A)
30 effluent from methanation section (I) to ATR section (II)
100 syngas stream

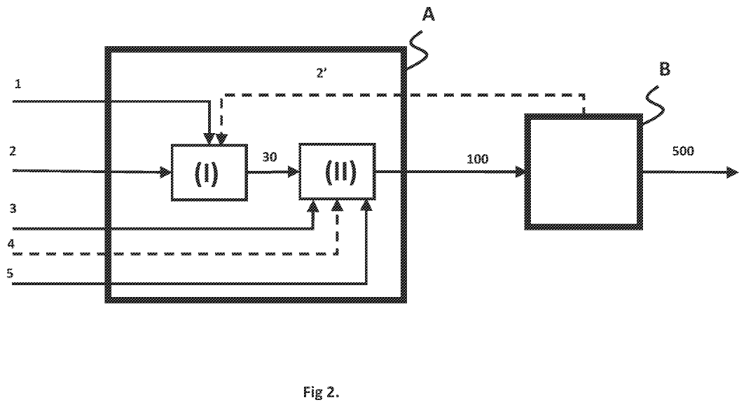

In FIG. 2, a synthesis stage B is also illustrated, which receives syngas stream 100 from the syngas stage A and converts it into product stream 500. References in this scheme are as for FIG. 1, with the additional reference 2′ to indicate a portion of the second feed (comprising carbon dioxide) from recycled from the synthesis stage B to the syngas stage A.

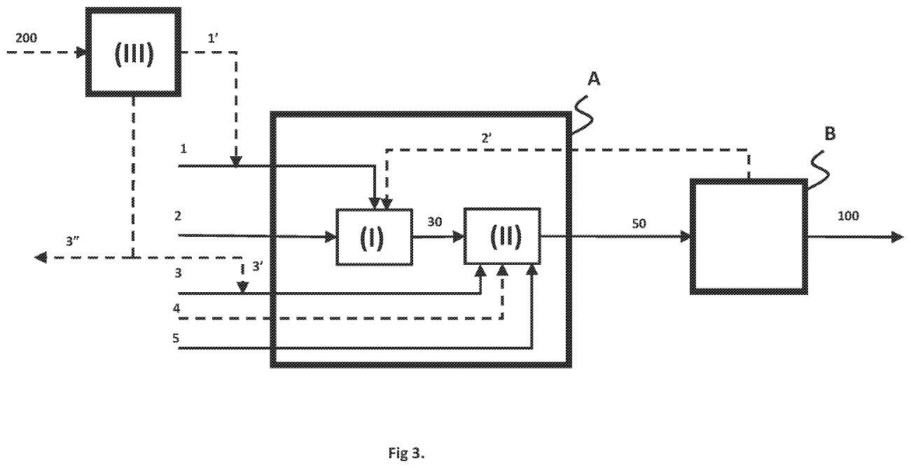

FIG. 3 shows a layout similar to that of FIG. 2, in which an electrolysis stage (III) is present. The electrolysis stage III separates a feed of water 200 into a part of third feed (comprising oxygen) from electrolysis stage 3′ and excess stream comprising oxygen from electrolysis stage 3″, as well as a part of the first feed comprising hydrogen 1′.

### EXAMPLES

In Table 1, some of the conceivable layouts of syngas production from primarily first feed (1) comprising H₂, second feed (2) comprising CO₂ and third feed (3) comprising O₂ are shown.

Optional use of fourth feed (4) comprising hydrocarbons is also possible. All examples comprise a methanation section with CH₄ concentration<20 vol %, followed by ATR section.

TABLE 1

| Parameters | Unit | C1 | C2 | C3 | C4 | C5 |
|---|---|---|---|---|---|---|
| H₂ content in first feed (1) | mol % | 99.0 | 99.0 | 99.0 | 99.0 | 99.0 |
| CO₂ content in second feed (2) | mol % | 99.9 | 99.9 | 99.9 | 99.9 | 99.9 |
| First feed (1)/second feed (2) | — | 3.47 | 3.17 | 3.47 | 3.17 | 3.19 |
| Third feed (3)/first feed (1) | — | 0.11 | 0.11 | 0.12 | 0.10 | 0.10 |
| Fourth feed (4)/second feed (2) | — | 0.33 | 0.30 | 0.33 | 0.00 | 0.33 |
| Fifth feed (5)/first feed (1) | — | 0.04 | 0.04 | 0.04 | 0.04 | 0.04 |
| H₂/CO in syngas product (100) | — | 2.00 | 2.00 | 2.00 | 2.11 | 2.00 |
| CO in syngas product (100)/total C in | % | 81.11 | 75.90 | 78.70 | 79.16 | 78.01 |
| feeds (external + internal streams) | | | | | | |
| Methanation section (I) inlet temp. | ° C. | 398 | 398 | 350 | 350 | 350 |
| Methanation section (I) outlet temp. | ° C. | 798 | 797 | 768 | 759 | 820 |
| Methanation section (I) outlet CH₄ conc. | vol % | 11.40 | 11.40 | 16.97 | 16.69 | 9.22 |
| Fraction of syngas to CO₂ removal | — | 0.50 | 0.30 | 0.30 | 0.40 | 0.33 |

In examples C1-C2, methanation section (I) doesn't have any effluent cooling within the section, between the methanation reactors, and effluent from methanation section (I) is sent directly to ATR section (II) along with some hydrocarbon comprising further feed (4). A part of the produced syngas is passed through a CO₂ removal stage, located downstream of ATR section (II). Recovered CO₂ is compressed and recycled to syngas stage (A) as a part of second feed (2).

In examples C3-C4, methanation section (I) consists of a couple of methanation units with intermediate effluent cooling. Some of water produced in the methanation unit is condensed out before directing it to last methanation unit. Effluent from methanation section (I) is sent directly to ATR section (II). A part of the produced syngas is passed through a CO₂ removal stage, located downstream of ATR section (II). Recovered CO₂ is compressed and recycled to syngas stage (A) as a part of second feed (2).

Interestingly, C4 demonstrates a particular example where there is no fourth feed (4) comprising hydrocarbon feeds.

In C5, methanation section comprises two methanation reactors—first an adiabatic one followed by a gas heated methanation reactor (gas heated using ATR effluent). However, unlike C3-C4, no water is condensed out between methanation reactors. The effluent from methanation section is fed directly to ATR section without any cooling.

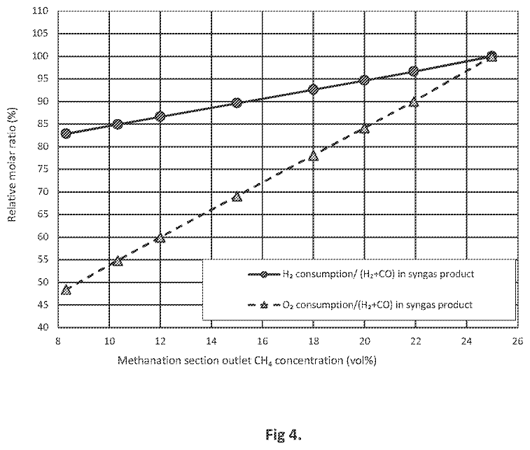

In FIG. 4, consumption of feeds (H₂ and O₂) relative to (H₂+CO) in syngas product from syngas stage to F-T synthesis are shown for different syngas stage layouts where feed compositions, second feed (2) flow and fourth feed (4) flow are kept the same. Only methanation section outlet temperature is changed. Additionally, first feed (1) flow is adjusted to keep a H₂/CO ratio of 2.0 in syngas product. The third feed (3) flow changes depending in the changes performed in the methanation section. From experience, it has been seen that final product from F-T synthesis (i.e. liquid fuels such as diesel, jet-fuel etc.) correlates very well with (H₂+CO) flow from syngas stage to synthesis stage. In other words, higher (H₂+CO) from syngas stage would result in more production of liquid. Therefore, comparison of first and third feed consumptions with respect (H₂+CO) in syngas among examples reflects effective utilization of feeds. Lower value of feed to (H₂+CO) in syngas indicates better utilization of feeds. For easier comparison, the consumption values are normalized with respect to Ex1.

Increase of relative H₂ consumption and O₂ consumption per (H₂+CO) in syngas (normalized based on Ex1) can solely be attributed to higher methanation section outlet CH₄ concentration, because both second feed (2) comprising CO₂ and fourth feed (4) have been kept the same. FIG. 4 clearly shows the more efficient utilization of first feed comprising H₂ and third feed comprising oxygen at lower extent of methanation. This is significant, as production of H₂ and O₂ are typically energy- and cost-intensive processes. The relationship set out herein is previously unknown, allowing new possibilities in syngas production.

1. A syngas stage (A) for a chemical plant, said syngas stage (A) comprising a methanation section (I) and an autothermal reforming (ATR) section (II); said syngas stage (A) comprising
a first feed comprising hydrogen to the syngas stage (A);
a second feed comprising carbon dioxide to the syngas stage (A);
a third feed comprising oxygen to the ATR section (II) in syngas stage (A);
wherein said syngas stage (A) is arranged to provide a syngas stream from said first, second and third feeds.

2. The syngas stage according to claim 1, wherein a part or all of the first feed is arranged to be fed to the methanation section (I); and a part or all of the second feed is arranged to be fed to the methanation section (I).

3. The syngas stage according to claim 1, wherein the methane content in the gas leaving the methanation section (I) is arranged to be less than 20%.

4. The syngas stage according to claim 1, wherein the methanation section (I) comprises one or more methanation units.

5. The syngas stage according to claim 1, wherein syngas stage (A) comprises a methanation section (I) and an ATR section (II), where the methanation section (I) comprises or consists of one or more adiabatic methanation units.

6. The syngas stage according to claim 1, wherein syngas stage (A) comprises a methanation section (I) and an ATR section (II), where the methanation section (I) comprises or consists of one or more heated methanation units.

7. The syngas stage according to claim 1, wherein syngas stage (A) comprises a methanation section (I) and an ATR section (II) wherein the methanation section (I) comprises at least one adiabatic methanation unit and at least one heated steam reforming unit.

8. The syngas stage according to claim 1, wherein the syngas stage (A) comprises or consists of a methanation section (I) arranged upstream an autothermal reforming (ATR) section (II).

9. The syngas stage according to claim 1, wherein a CO2 removal stage is arranged downstream the syngas stage (A).

10. The syngas stage according to claim 9, wherein a part or all of the CO2 removed in the CO2 removal stage is arranged to be recycled to the syngas stage (A) as part of the second feed comprising carbon dioxide.

11. The syngas stage according to claim 1, further comprising an electrolyser stage arranged to convert water or steam into at least a hydrogen-containing stream and an oxygen-containing stream, and wherein at least a part of said hydrogen-containing stream from the electrolyser stage is fed to the syngas stage (A) as part or all of said first feed (1) and/or wherein at least a part of said oxygen-containing stream from the electrolyser stage is fed to the syngas stage (A) as part or all said third feed (3).

12. The syngas stage according to claim 1, wherein the syngas stage comprises a fourth feed comprising hydrocarbons to said methanation section (I) and/or to said ATR section (II).

13. The syngas stage according to claim 12, wherein a part or all of the fourth feed comprising hydrocarbons is arranged to be fed to the ATR section (II).

14. The syngas stage according to claim 12, wherein the ratio of moles of carbon in the fourth feed comprising hydrocarbons, when external to the syngas stage, to the moles of carbon in the second feed (2) is less than 0.50.

15. The syngas stage according to claim 1, further comprising a fifth feed of steam to the methanation section (I) and/or the ATR section (II).

16. The syngas stage according to claim 1, wherein the ratio of H_{2:}CO₂ provided at the syngas stage inlet is between 1.0-9.07.

17. The syngas stage according to claim 1, wherein the syngas stream has a hydrogen/carbon monoxide ratio in the range 1.0-4.0.

18. A chemical plant comprising the syngas stage according to claim 12, and a synthesis stage (B), wherein said syngas stage (A) is arranged to feed said syngas stream (100) to said synthesis stage, said synthesis stage being a Fischer-Tropsch (F-T) stage, being arranged to provide at least a product stream and a hydrocarbon-containing off-gas stream, wherein at least a portion of the hydrocarbon-containing off-gas stream from the F-T stage is arranged to be fed to the syngas stage, as all or part of the fourth feed comprising hydrocarbons.

19. A method for producing a syngas stream, said method comprising the steps of:
providing a syngas stage (A) as defined in any one of the preceding claims;
supplying a first feed comprising hydrogen to the syngas stage (A);
supplying a second feed comprising carbon dioxide to the syngas stage (A);
supplying a third feed comprising oxygen to the ATR section (II); and
optionally, supplying a fourth feed (4) comprising hydrocarbons to said methanation section (I) and/or to said ATR section (II);
converting said first, second, third and—optionally, fourth—feeds in said syngas stage (A) to a syngas stream.

20. The method according to claim 19, wherein said syngas stage comprises a methanation section (I) and an ATR section (II) and wherein no water condensation takes place in the methanation section (I).

21. The method according to claim 19, wherein said syngas stage comprises a methanation section (I) and an ATR section (II) wherein the methanation section (I) comprises or consists of one or more adiabatic methanation units, and wherein the temperature of the gas at the exit of the adiabatic methanation unit is greater than 700° C.

22. The method according to claim 19, wherein said syngas stage comprises a methanation section (I) and an ATR section (II) wherein the methanation section (I) comprises or consists of an adiabatic methanation unit, and wherein no active cooling of the gas exiting the adiabatic methanation unit takes place before said gas is directed to the ATR section (II).

23. The method according to claim 19, wherein the methanation section (I) comprises or consists of one or more methanation units, and wherein the gas temperature at the inlet to the first methanation unit in the methanation section is >350° C.

24. The method according to claim 19, wherein a CO2 removal stage is arranged downstream the syngas stage and wherein CO2 is removed from syngas stream by means of said CO2 removal stage.

25. The method according to claim 24, wherein a part or all of the CO2 removed in the CO2 removal stage is recycled to syngas stage (A) as part of said second feed comprising carbon dioxide

26. The method according to claim 19, wherein the methane content in the gas leaving the methanation section (I) is less than 20%.
